(19) United States
(12) Patent Application Publication
(10) Pub. No.: US 2026/0165862 A1
(43) Pub. Date: Jun. 18, 2026

(54) STENT SYSTEM

(71) Applicant: BOSTON SCIENTIFIC SCIMED, INC., Maple Grove, MN (US)

(72) Inventors: DAVID GILES, Tuam (IE); FIONN STAPLETON, Oranmore (IE); DANIEL TUCK, Galway (IE); GARY GILMARTIN, Foxford (IE); SHANE O'KEEFFE, Tuam (IE); KEITH O'REILLY, Galway (IE); THOMAS MARTIN KEATING, Tuam (IE); JOHN THOMAS O'DRISCOLL, Galway (IE)

(73) Assignee: BOSTON SCIENTIFIC SCIMED, INC., Maple Grove, MN (US)

(21) Appl. No.: 19/420,621

(22) Filed: Dec. 15, 2025

Related U.S. Application Data
(60) Provisional application No. 63/734,469, filed on Dec. 16, 2024.

(51) Int. Cl. A61F 2/958

(52) U.S. Cl. CPC A61F 2/958; A61F 2002/9583

(57) ABSTRACT

A stent system includes a self-expanding endoprosthesis and a stent delivery system including an elongate shaft having an inflatable balloon disposed proximate a distal end, and an endoprosthesis retention element disposed around the self-expanding endoprosthesis and the inflatable balloon. The retention element is configured to constrain the endoprosthesis in a radially collapsed configuration. The retention element is configured to fracture upon inflation of the balloon. The endoprosthesis is configured to exert a first radially outward force in the radially collapsed configuration. The retention element is configured to fracture when a second radially outward force greater than the first is applied to the retention element. The retention element includes a tubular sleeve disposed radially outward of and in intimate contact with the endoprosthesis, wherein the tubular sleeve is configured to break apart when the balloon is shifted toward the inflated configuration.

### CROSS REFERENCE TO RELATED APPLICATIONS

The present application claims the benefit of U.S. Provisional Patent Application Ser. No. 63/734,469, filed Dec. 16, 2024, the disclosure of which is incorporated herein by reference.

### TECHNICAL FIELD

The present disclosure pertains to medical devices, methods for manufacturing medical devices, and uses thereof. More particularly, the present disclosure pertains to a system including an endoprosthesis or stent for implantation in a body lumen and/or a stent delivery system for implanting the endoprosthesis or stent in a body lumen, and associated methods.

### BACKGROUND

An endoprosthesis may be used in the treatment of body lumens. One type of endoprosthesis used in the repair and/or treatment of diseases in various body lumens is a stent. A stent is a generally longitudinal tubular device formed of biocompatible material which is useful to open and support various lumens in the body. For example, stents may be used in the vascular system, urogenital tract, gastrointestinal tract, esophageal tract, tracheal/bronchial tubes, and bile duct, as well as in a variety of other applications in the body.

In some instances, smooth and accurate deployment of self-expanding endoprostheses or stents may be difficult due to friction between moving elements of the delivery system, or other reasons. In some instances, it may be desirable to limit foreshortening. In some instances, it may be desirable to reduce friction between moving elements of a delivery system. In some instances, it may be desirable to simplify the delivery system. In some instances, it may be desirable to improve deployment accuracy.

Of the known medical devices and methods, each has certain advantages and disadvantages. There is an ongoing need to provide alternative medical devices as well as alternative methods for manufacturing and using medical devices.

### SUMMARY

In one example, a stent system may comprise a self-expanding endoprosthesis configured to shift from a radially collapsed configuration to a radially expanded configuration, and a stent delivery system. The stent delivery system may comprise an elongate shaft comprising an inflatable balloon disposed proximate a distal end of the elongate shaft, wherein the inflatable balloon may be configured to shift between a deflated configuration and an inflated configuration, wherein the self-expanding endoprosthesis may be configured to be disposed on the inflatable balloon in the radially collapsed configuration with the inflatable balloon in the deflated configuration, and an endoprosthesis retention element disposed around the self-expanding endoprosthesis in the radially collapsed configuration and the inflatable balloon in the deflated configuration. The endoprosthesis retention element may be configured to constrain the self-expanding endoprosthesis in the radially collapsed configuration. The endoprosthesis retention element may be configured to fracture upon inflation of the inflatable balloon to the inflated configuration.

In addition, or alternatively, to any example disclosed herein, the endoprosthesis retention element has an overall length that is less than 125% of a length of the self-expanding endoprosthesis.

In addition, or alternatively, to any example disclosed herein, a radially outermost extent of the inflatable balloon in the inflated configuration is less than a radially innermost extent of the self-expanding endoprosthesis in the radially expanded configuration.

In addition, or alternatively, to any example disclosed herein, shifting the inflatable balloon to the inflated configuration does not shift the self-expanding endoprosthesis to the radially expanded configuration.

In addition, or alternatively, to any example disclosed herein, an inner diameter of the endoprosthesis retention element is less than a radially outermost extent of the inflatable balloon in the inflated configuration.

In addition, or alternatively, to any example disclosed herein, the endoprosthesis retention element does not extend to a proximal end of the elongate shaft.

In addition, or alternatively, to any example disclosed herein, the stent delivery system is devoid of an actuation mechanism configured to move the endoprosthesis retention element axially relative to the elongate shaft without moving the elongate shaft.

In addition, or alternatively, to any example disclosed herein, and in a second example, a stent system may comprise a self-expanding endoprosthesis configured to shift from a radially collapsed configuration to a radially expanded configuration, wherein the self-expanding endoprosthesis may be configured to exert a first radially outward force in the radially collapsed configuration, and a stent delivery system. The stent delivery system may comprise an elongate shaft comprising an inflatable balloon disposed proximate a distal end of the elongate shaft, wherein the inflatable balloon may be configured to shift between a deflated configuration and an inflated configuration, wherein the self-expanding endoprosthesis may be configured to be disposed on the inflatable balloon in the radially collapsed configuration with the inflatable balloon in the deflated configuration, and an endoprosthesis retention element disposed around the self-expanding endoprosthesis in the radially collapsed configuration and the inflatable balloon in the deflated configuration. The endoprosthesis retention element may be configured to constrain the self-expanding endoprosthesis in the radially collapsed configuration. The endoprosthesis retention element may be configured to fracture when a second radially outward force greater than the first radially outward force is applied to an inner surface of the endoprosthesis retention element.

In addition, or alternatively, to any example disclosed herein, the endoprosthesis retention element is bioabsorbable.

In addition, or alternatively, to any example disclosed herein, fracturing the endoprosthesis retention element is configured to completely detach the endoprosthesis retention element from a remainder of the stent delivery system.

In addition, or alternatively, to any example disclosed herein, the stent delivery system is devoid of an actuation mechanism configured to move the endoprosthesis retention element axially relative to the self-expanding endoprosthesis.

In addition, or alternatively, to any example disclosed herein, the inflatable balloon is configured to apply the second radially outward force against the inner surface of the endoprosthesis retention element.

In addition, or alternatively, to any example disclosed herein, the inflatable balloon is configured to apply the second radially outward force against the inner surface of the endoprosthesis retention element when the inflatable balloon is in the inflated configuration.

In addition, or alternatively, to any example disclosed herein, the inflatable balloon is incapable of fully shifting the self-expanding endoprosthesis to the radially expanded configuration.

In addition, or alternatively, to any example disclosed herein, and in a third example, a stent system may comprise a self-expanding endoprosthesis configured to shift from a radially collapsed configuration to a radially expanded configuration, and a stent delivery system comprising an elongate shaft comprising an inflatable balloon disposed proximate a distal end of the elongate shaft, wherein the inflatable balloon may be configured to shift between a deflated configuration and an inflated configuration, wherein the self-expanding endoprosthesis may be configured to be disposed on the inflatable balloon in the radially collapsed configuration with the inflatable balloon in the deflated configuration, and an endoprosthesis retention element comprising a tubular sleeve disposed radially outward of and in intimate contact with the self-expanding endoprosthesis, wherein the endoprosthesis retention element may be configured to constrain the self-expanding endoprosthesis in the radially collapsed configuration on the inflatable balloon when the inflatable balloon is in the deflated configuration. The tubular sleeve may be configured to break apart when the inflatable balloon is shifted from the deflated configuration toward the inflated configuration.

In addition, or alternatively, to any example disclosed herein, the tubular sleeve is axially immovable relative to the self-expanding endoprosthesis via manipulation of the stent delivery system.

In addition, or alternatively, to any example disclosed herein, the tubular sleeve is not fixedly attached directly to any other structure of the stent system.

In addition, or alternatively, to any example disclosed herein, the self-expanding endoprosthesis is formed from a single filament of material.

In addition, or alternatively, to any example disclosed herein, the tubular sleeve is configured to break apart into a plurality of fragments when the inflatable balloon is shifted from the deflated configuration toward the inflated configuration.

In addition, or alternatively, to any example disclosed herein, the tubular sleeve is configured to retain its structural integrity until the tubular sleeve is broken apart as a result of shifting the inflatable balloon from the deflated configuration toward the inflated configuration.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIGS. 1-2 schematically illustrate selected aspects of a prior art stent delivery system;

FIG. 3 is a flat pattern view illustrating selected aspects of an endoprosthesis in accordance with the disclosure;

FIG. 4 is a partial cross-sectional view schematically illustrating selected aspects of a stent system according to the disclosure disposed within a body lumen;

FIGS. 5-7 are partial cross-sectional views schematically illustrating selected aspects related to deployment of an endoprosthesis using the stent system of FIG. 4; and

FIGS. 8-9 are partial cross-sectional views schematically illustrating selected aspects related to the endoprosthesis deployed within the body lumen post-deployment.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

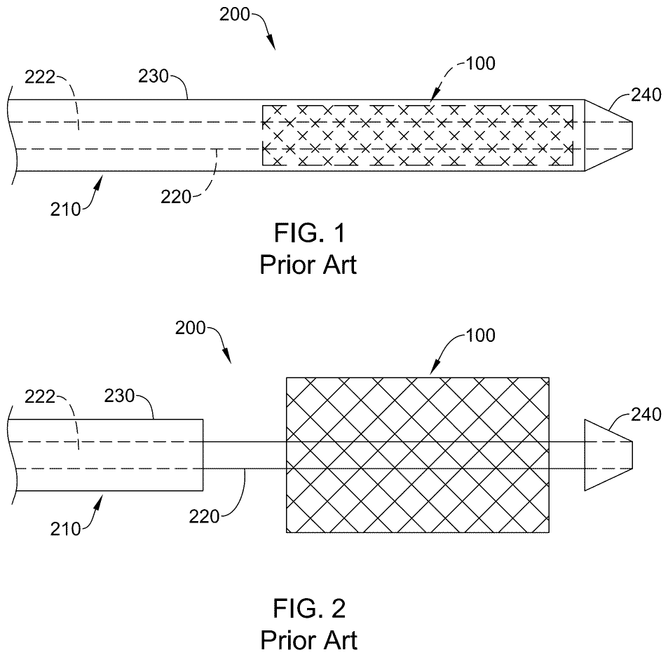

FIGS. 1-2 illustrate selected aspects of a prior art stent system 200 comprising a self-expanding stent 100 and a delivery device 210. The delivery device 210 comprises an inner tubular member 220 and an outer tubular member 230. The self-expanding stent 100 is disposed within an annular space between the inner tubular member 220 and the outer tubular member 230 (e.g., within a lumen of the outer tubular member 230 and around the inner tubular member 220) in a delivery configuration, as seen in FIG. 1. The inner tubular member 220 and the outer tubular member 230 are axially movable relative to each other to load the self-expanding stent 100 into, and deploy the self-expanding stent 100 from, the delivery device 210. The inner tubular member 220 comprises a guidewire lumen 222 extending therein. The delivery device 210 comprises a distal tip 240 disposed at a distal end of the inner tubular member 220. The self-expanding stent 100 is configured to self-expand from the delivery configuration (e.g., FIG. 1) to a deployed configuration, as seen in FIG. 2, when unconstrained by the outer tubular member 230. In some situations, slack in the delivery device 210 may cause variation in and/or inaccurate deployment of the self-expanding stent 100. In some situations, friction between the inner tubular member 220 and the outer tubular member 230 may cause variation in and/or inaccurate deployment of the self-expanding stent 100. In some situations, the distal tip 240 may move or “jump” forward (e.g., distally) during deployment of self-expanding stent 100, resulting in variation in and/or inaccurate deployment of the self-expanding stent 100. Discussed herein is a stent system intended, designed, and/or adapted to address one or more of these situations to improve deployment accuracy and reduce variation in deployment of a self-expanding endoprosthesis.

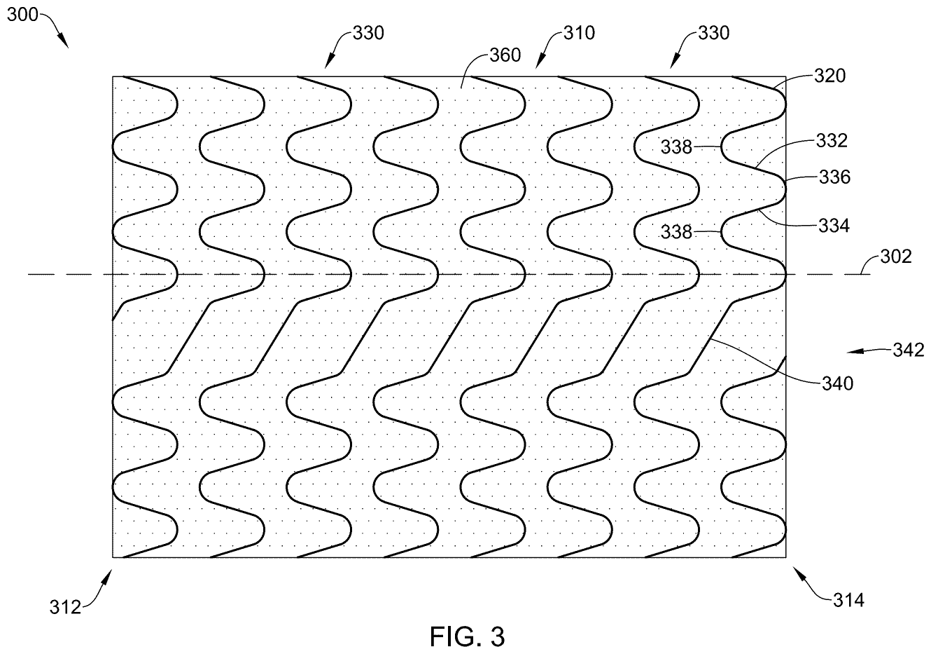

FIG. 3 illustrates selected aspects of one example of a self-expanding endoprosthesis 300, such as a stent, usable with the current disclosure. For reference, FIG. 3 illustrates the self-expanding endoprosthesis 300 in a flat pattern view. The self-expanding endoprosthesis 300 may comprise a body portion 310 extending axially from a first end 312 to a second end 314 along a central longitudinal axis 302. In some embodiments, the first end 312 and the second end 314 may define a length of the self-expanding endoprosthesis 300 and/or the body portion 310. In at least some embodiments, the body portion 310 may be tubular. The body portion 310 may define a lumen extending therethrough from the first end 312 to the second end 314.

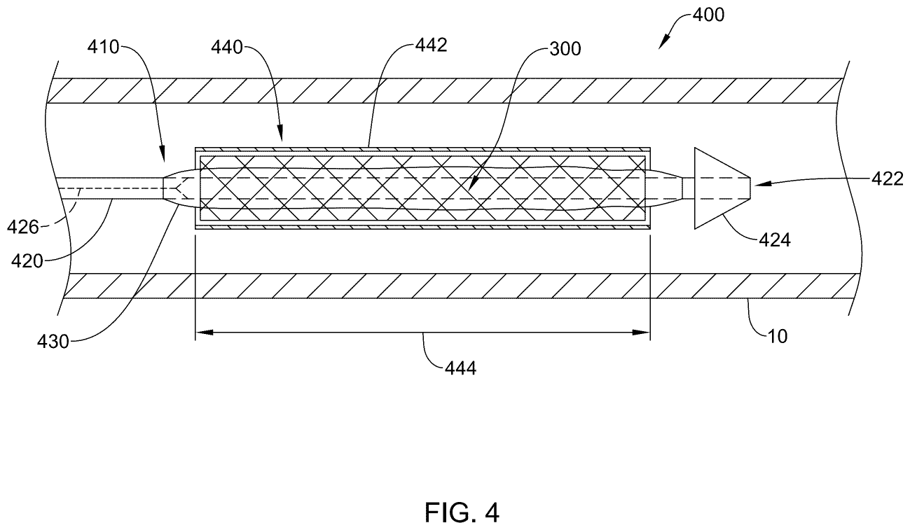

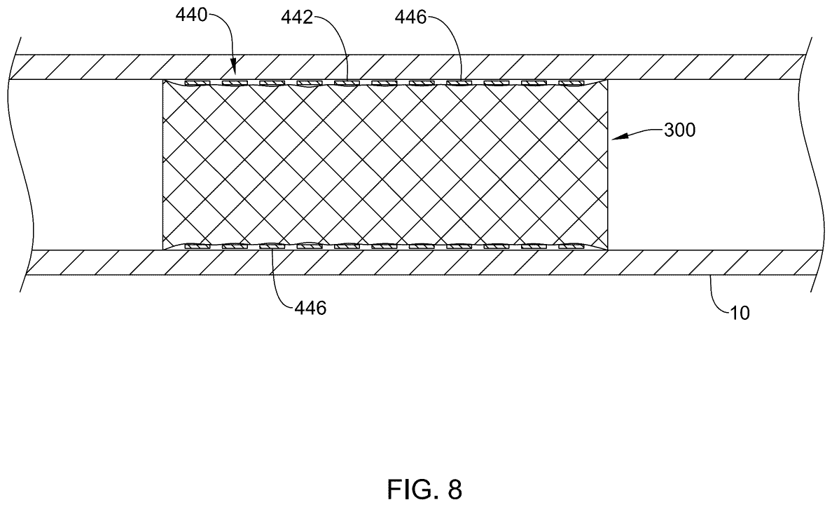

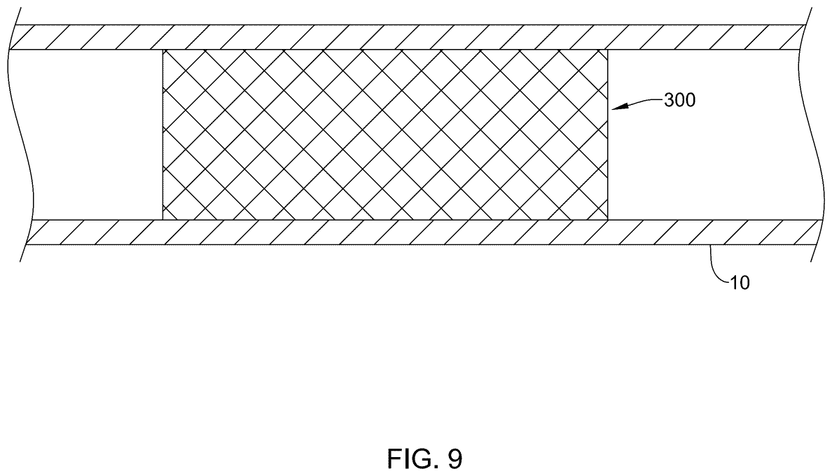

The self-expanding endoprosthesis 300 and/or the body portion 310 thereof may be configured to shift from a radially collapsed configuration (e.g., FIG. 4) toward and/or to a radially expanded configuration (e.g., FIGS. 8-9). In at least some embodiments, the self-expanding endoprosthesis 300 may be configured to self-expand from the radially collapsed configuration toward and/or to the radially expanded configuration. In some embodiments, the self-expanding endoprosthesis 300 may be self-biased toward and/or to the radially expanded configuration. In some embodiments, the self-expanding endoprosthesis 300 may be configured to exert a first radially outward force in the radially collapsed configuration. In at least some embodiments, the first radially outward force may be sufficient to shift the self-expanding endoprosthesis 300 from the radially collapsed configuration to the radially expanded configuration when the self-expanding endoprosthesis 300 is unconstrained. In some embodiments, the first radially outward force may be sufficient to shift the self-expanding endoprosthesis 300 from the radially collapsed configuration to the radially expanded configuration without assistance from any other structure and/or without any additional radially outward force added to the first radially outward force when the self-expanding endoprosthesis 300 is unconstrained.

In some embodiments, the self-expanding endoprosthesis 300 may comprise a flared first end portion (not shown) and/or a flared second end portion (not shown), wherein an outer diameter of the flared first end portion is greater than an outer diameter of the body portion 310 and/or an outer diameter of the flared second end portion is greater than the outer diameter of the body portion 310. Other configurations are also contemplated. The flared first end portion may be disposed proximate and/or adjacent the first end 312. In some embodiments, the flared first end portion may extend from the first end 312 to the body portion 310. The flared second end portion may be disposed proximate and/or adjacent the second end 314. In some embodiments, the flared second end portion may extend from the second end 314 to the body portion 310.

In some embodiments, the outer diameter of the body portion 310 may be generally uniform and/or generally constant along the length of the self-expanding endoprosthesis 300 and/or the body portion 310. In some embodiments, the outer diameter of the body portion 310 may be generally uniform and/or generally constant along the length of the body portion 310 except for the flared first end portion and/or the flared second end portion, where present. In some embodiments, the outer diameter of the body portion 310 may be generally uniform and/or generally constant along the length of the body portion 310 from the flared first end portion to the flared second end portion, where present. In some embodiments, the outer diameter of the body portion 310 may be between about 4 millimeters and about 40 millimeters, between about 6 millimeters and about 35 millimeters, between about 8 millimeters and about 30 millimeters, between about 10 millimeters and about 25 millimeters, etc. Other configurations are also contemplated.

In some embodiments, the self-expanding endoprosthesis 300 and/or the body portion 310 may be formed from a single filament 320 of material (e.g., a single wire, etc.) extending from the first end 312 to the second end 314. In some embodiments, the flared first end portion and/or the flared second end portion, where present, may be formed from the single filament 320. The single filament 320 may extend circumferentially around the central longitudinal axis 302 and/or the single filament 320 may encircle the central longitudinal axis 302. The single filament 320 is and/or consists of only one filament of material (e.g., only one wire, etc.).

In some embodiments, the single filament 320 may form a plurality of circumferential segments 330 extending non-helically around the central longitudinal axis 302. In some embodiments, the plurality of circumferential segments 330 may be substantially parallel to each other. In some embodiments, the plurality of circumferential segments 330 may extend helically around the central longitudinal axis 302. In some embodiments, each circumferential segment of the plurality of circumferential segments 330 may form a ring extending around the central longitudinal axis 302. Some suitable but non-limiting examples of materials for the self-expanding endoprosthesis 300 and/or the single filament 320, such as metallic materials, composite materials, shape memory materials, combinations thereof, etc., are discussed below. In one non-limiting example, the self-expanding endoprosthesis 300 and/or the single filament 320 may be formed from nickel-titanium alloy (e.g., nitinol). In another non-limiting example, the self-expanding endoprosthesis 300 and/or the single filament 320 may be formed from nickel-titanium alloy with a platinum core. Other configurations and/or materials are also contemplated.

In some embodiments, the plurality of circumferential segments 330 may comprise a first end circumferential segment forming the first end 312, a second end circumferential segment forming the second end 314, and at least one medial circumferential segment disposed axially between the first end circumferential segment and the second end circumferential segment. In some embodiments, the at least one medial circumferential segment may comprise one circumferential segment, two circumferential segments, three circumferential segments, etc. up to a desired number of circumferential segments producing a desired overall length for the self-expanding endoprosthesis 300.

In some embodiments, each circumferential segment of the plurality of circumferential segments 330 may comprise an undulating arrangement (e.g., a sinusoidal arrangement) of first struts 332 and second struts 334 defining peaks 336 and valleys 338. In at least some embodiments, within any particular circumferential segment of the plurality of circumferential segments 330, the first struts 332 have a first length defined by the peaks 336 and the valleys 338 and the second struts 334 have a second length defined by the peaks 336 and the valleys 338, wherein the first length is equal to the second length. In some embodiments, the first length and the second length may be uniform and/or constant among the plurality of circumferential segments 330. In some alternative embodiments, the first length and the second length may vary among the plurality of circumferential segments 330. In some embodiments, the first length and/or the second length may be between about 1 millimeter and about 15 millimeters. In some embodiments, the first length and/or the second length may be between about 3 millimeters and about 12 millimeters. In some embodiments, the first length and/or the second length may be between about 5 millimeters and about 10 millimeters. Other configurations are also contemplated.

In some embodiments, the peaks 336 and the valleys 338 of adjacent circumferential segments of the plurality of circumferential segments 330 may be aligned with each other axially along the length of the body portion 310 and/or the self-expanding endoprosthesis 300. For example, the peaks 336 of one circumferential segment of the plurality of circumferential segments 330 may be aligned axially with the peaks 336 of an immediately adjacent circumferential segment of the plurality of circumferential segments 330. Similarly, the valleys 338 of one circumferential segment of the plurality of circumferential segments 330 may be aligned axially with the valleys 338 of an immediately adjacent circumferential segment of the plurality of circumferential segments 330. In some embodiments, the peaks 336 and the valleys 338 of adjacent circumferential segments of the plurality of circumferential segments 330 may be aligned parallel to the central longitudinal axis 302. Other configurations are also contemplated.

In some embodiments, the peaks 336 and the valleys 338 of each circumferential segment of the plurality of circumferential segments 330 may be aligned with each other axially along the length of the body portion 310 and/or the self-expanding endoprosthesis 300. For example, the peaks 336 of each circumferential segment of the plurality of circumferential segments 330 may be aligned axially with the peaks 336 of each other circumferential segment of the plurality of circumferential segments 330. Similarly, the valleys 338 of each circumferential segment of the plurality of circumferential segments 330 may be aligned axially with the valleys 338 of each other circumferential segment of the plurality of circumferential segments 330. In some embodiments, the peaks 336 and the valleys 338 of each circumferential segment of the plurality of circumferential segments 330 may be aligned parallel to the central longitudinal axis 302. Other configurations are also contemplated.

In some embodiments, each circumferential segment of the plurality of circumferential segments 330 may be connected to an axially adjacent circumferential segment of the plurality of circumferential segments 330 by a transition segment 340 of the single filament 320. In at least some embodiments, each circumferential segment of the plurality of circumferential segments 330 may be discontinuous and/or may extend around less than a full circumference of the self-expanding endoprosthesis 300 and/or the body portion 310. Instead, each circumferential segment of the plurality of circumferential segments 330 may have a first end and a second end, wherein the transition segment 340 extends between a first end of one circumferential segment of the plurality of circumferential segments 330 and a second end of an immediately adjacent circumferential segment of the plurality of circumferential segments 330 (or the transition segment 340 extends between a second end of one circumferential segment of the plurality of circumferential segments 330 and a first end of an immediately adjacent circumferential segment of the plurality of circumferential segments 330).

The transition segment 340 of the single filament 320 may be oriented at an oblique angle relative to the central longitudinal axis 302 in the side view and/or the flat pattern view of the body portion 310 and/or the self-expanding endoprosthesis 300. In some embodiments, the oblique angle may be between about 30 degrees and about 90 degrees. In some embodiments, the oblique angle may be between about 35 degrees and about 75 degrees. In some embodiments, the oblique angle may be between about 40 degrees and about 60 degrees. Other configurations are also contemplated. In some embodiments, the transition segment 340 may have a length between about 5 millimeters and about 20 millimeters. In some embodiments, the transition segment 340 may have a length between about 8 millimeters and about 17 millimeters. In some embodiments, the transition segment 340 may have a length between about 10 millimeters and about 15 millimeters. Other configurations are also contemplated.

In some embodiments, each circumferential segment of the plurality of circumferential segments 330 may be connected to an axially adjacent circumferential segment of the plurality of circumferential segments 330 by a transition segment 340 of the single filament 320 disposed nonparallel to the central longitudinal axis 302, the first struts 332, and the second struts 334 in the side view and/or the flat pattern view of the body portion 310 and/or the self-expanding endoprosthesis 300. Other configurations are also contemplated.

In some embodiments, the transition segment 340 extending and/or disposed between axially adjacent circumferential segments of the plurality of circumferential segments 330 may extend between a valley (ref. 338) of one circumferential segment and a peak (ref. 336) of an immediately axially adjacent circumferential segment (or between a peak of one circumferential segment and a valley of an immediately axially adjacent circumferential segment). As illustrated, the transition segment(s) 340 extends from a peak (ref. 336) of a first circumferential segment disposed closer to the first end 312 to a valley (ref. 338) of a second circumferential segment immediately axially adjacent the first circumferential segment that is disposed closer to the second end 314 than the first circumferential segment. Other configurations are also contemplated. In some embodiments, the transition segment 340 may be disposed nonparallel with the first struts 332 and the second struts 334 of one circumferential segment (e.g., the first circumferential segment) and the first struts 332 and the second struts 334 of the immediately axially adjacent circumferential segment (e.g., the second circumferential segment).

In some embodiments, all transition segments (e.g., an array of transition segments consisting of each transition segment 340 connecting and/or extending between adjacent circumferential segments of the plurality of circumferential segments 330) of the single filament 320 may collectively define a transition zone 342. In some embodiments, the transition zone 342 may extend and/or be aligned parallel to the central longitudinal axis 302, as seen in FIG. 3. In some embodiments, the oblique angle may be uniform along and/or within the transition zone 342. In some embodiments, each transition segment 340 of the single filament 320 may be aligned axially along the length of the body portion 310 and/or the self-expanding endoprosthesis 300. In some embodiments, the transition zone 342 may define a preferential bending plane for the self-expanding endoprosthesis 300 and/or the body portion 310 (e.g., the self-expanding endoprosthesis 300 and/or the body portion 310 may be configured to bend more easily along and/or within the transition zone 342 than in other directions). In some alternative embodiments, the oblique angle may vary along and/or within the transition zone 342. In some embodiments, the transition zone 342 may extend and/or be aligned helically along and/or around the central longitudinal axis 302.

In some embodiments, the peaks 336 and the valleys 338 of adjacent circumferential segments of the plurality of circumferential segments 330 may extend and/or be aligned helically along the length of the body portion 310 and/or the self-expanding endoprosthesis 300. In some embodiments, the peaks 336 and the valleys 338 of adjacent circumferential segments of the plurality of circumferential segments 330 may extend and/or be aligned helically along and/or around the central longitudinal axis 302. In some embodiments, the peaks 336 and the valleys 338 of each circumferential segment of the plurality of circumferential segments 330 may extend and/or be aligned helically along the length of the body portion 310 and/or the self-expanding endoprosthesis 300. In some embodiments, the peaks 336 and the valleys 338 of each circumferential segment of the plurality of circumferential segments 330 may extend and/or be aligned helically along and/or around the central longitudinal axis 302. Other configurations are also contemplated.

In some embodiments, the self-expanding endoprosthesis 300 and/or the body portion 310 may comprise one or more anti-migration elements (not shown) disposed along the length of the self-expanding endoprosthesis 300 and/or the body portion 310. In some embodiments, at least one circumferential segment of the plurality of circumferential segments 330 may comprise an anti-migration element projecting radially outward from the at least one circumferential segment and/or an outer surface of the self-expanding endoprosthesis 300 and/or the body portion 310 in the radially expanded configuration. In some embodiments, at least one circumferential segment of the plurality of circumferential segments 330 may comprise a plurality of anti-migration elements projecting radially outward from the at least one circumferential segment and/or the outer surface of the self-expanding endoprosthesis 300 and/or the body portion 310 in the radially expanded configuration.

In some embodiments, anti-migration element(s) positioned proximate the first end 312 may project radially outward and toward the second end 314 (e.g., at a non-zero angle relative to the outer surface of the self-expanding endoprosthesis 300 and/or the body portion 310) and/or anti-migration element(s) positioned proximate the second end 314 may project radially outward and toward the first end 312 (e.g., at a non-zero angle relative to the outer surface of the self-expanding endoprosthesis 300 and/or the body portion 310). In some embodiments, anti-migration element(s) positioned proximate the first end 312 may project radially outward and toward the first end 312 (e.g., at a non-zero angle relative to the outer surface of the self-expanding endoprosthesis 300 and/or the body portion 310) and/or anti-migration element(s) positioned proximate the second end 314 may project radially outward and toward the second end 314 (e.g., at a non-zero angle relative to the outer surface of the self-expanding endoprosthesis 300 and/or the body portion 310). In some embodiments, some anti-migration elements positioned proximate the first end 312 may project toward the first end 312 and some may project toward the second end 314 (e.g., at a non-zero angle relative to the outer surface of the self-expanding endoprosthesis 300 and/or the body portion 310), and/or some anti-migration elements positioned proximate the second end 314 may project toward the first end 312 and some may project toward the second end 314 (e.g., at a non-zero angle relative to the outer surface of the self-expanding endoprosthesis 300 and/or the body portion 310). Other configurations are also contemplated.

In some embodiments, the self-expanding endoprosthesis 300 may comprise a polymeric covering 360, shown in the figures via dotted shading, coupled to the body portion 310, the single filament 320, and/or the plurality of circumferential segments 330. In some embodiments, the polymeric covering 360 may be fixedly attached to the body portion 310, the single filament 320, and/or the plurality of circumferential segments 330.

In some embodiments, the polymeric covering 360 may extend along an inner surface of the self-expanding endoprosthesis 300, the body portion 310, the single filament 320, and/or the plurality of circumferential segments 330. In some embodiments, the polymeric covering 360 may extend along an outer surface of the self-expanding endoprosthesis 300, the body portion 310, the single filament 320, and/or the plurality of circumferential segments 330. In at least some embodiments, the body portion 310, the single filament 320, and/or the plurality of circumferential segments 330 may be embedded within the polymeric covering 360. In some embodiments, the anti-migration element, the plurality of anti-migration elements, etc. may be embedded within the polymeric covering 360. Other configurations, including combinations thereof, are also contemplated. Some suitable but non-limiting examples of polymeric materials for the polymeric covering 360 are discussed below.

In some embodiments, the body portion 310 and/or the self-expanding endoprosthesis 300 may be configured to minimize and/or avoid foreshortening and/or a change in the length of the body portion 310 and/or the self-expanding endoprosthesis 300 as the body portion 310 and/or the self-expanding endoprosthesis 300 shifts between the radially collapsed configuration and the radially expanded configuration. In some embodiments, the length of the body portion 310 and/or the self-expanding endoprosthesis 300 from the first end 312 to the second end 314 may be configured to change by less than 10% when shifting between the radially collapsed configuration and the radially expanded configuration. In some embodiments, the length of the body portion 310 and/or the self-expanding endoprosthesis 300 from the first end 312 to the second end 314 may be configured to change by less than 7.5% when shifting between the radially collapsed configuration and the radially expanded configuration. In some embodiments, the length of the body portion 310 and/or the self-expanding endoprosthesis 300 from the first end 312 to the second end 314 may be configured to change by less than 5% when shifting between the radially collapsed configuration and the radially expanded configuration. In some embodiments, the length of the body portion 310 and/or the self-expanding endoprosthesis 300 from the first end 312 to the second end 314 may be configured to change by less than 2.5% when shifting between the radially collapsed configuration and the radially expanded configuration. In some embodiments, the length of the body portion 310 and/or the self-expanding endoprosthesis 300 from the first end 312 to the second end 314 may be configured to change by less than 1% when shifting between the radially collapsed configuration and the radially expanded configuration. Other configurations are also contemplated.

FIG. 4 schematically illustrates selected aspects of a stent system 400 in accordance with the disclosure. In some embodiments, the stent system 400 may comprise a self-expanding endoprosthesis, such as but not limited to the self-expanding endoprosthesis 300 described herein, configured to shift from a radially collapsed configuration to a radially expanded configuration. In some embodiments, the stent system 400 may comprise a stent delivery system 410. The stent delivery system 410 may be configured to deliver the self-expanding endoprosthesis 300 to, and/or may be configured to deploy the self-expanding endoprosthesis 300 within, a body lumen 10. The body lumen 10 may be any one of a variety of body lumens, such as but not limited to a blood vessel, esophageal tract, digestive tract, biliary tract, renal tract, etc., as is known in the art. Other configurations are also contemplated.

In some embodiments, the stent delivery system 410 may be a standalone stent delivery system and/or device configured to be advanced to the body lumen 10 on its own (e.g., without the aid of guide sheath, access sheath, etc.). In some embodiments, the stent delivery system 410 may be configured to be advanced to the body lumen 10 within and/or through a tubular member (such as a guide sheath, an access sheath, etc.), a catheter, an endoscope, etc. Other configurations are also contemplated.

In some embodiments, the stent delivery system 410 may comprise an elongate shaft 420 comprising an inflatable balloon 430 disposed proximate a distal end of the elongate shaft 420. The inflatable balloon 430 may be configured to shift between a deflated configuration and an inflated configuration. In at least some embodiments, the inflatable balloon 430 may be configured to be disposed in the deflated configuration during advancement and/or navigation of the stent delivery system 410 to the body lumen 10.

In some embodiments, the elongate shaft 420 may comprise a guidewire lumen 422 extending therein. In some embodiments, the elongate shaft 420 may comprise a nosecone 424 fixedly attached at the distal end of the elongate shaft 420. In some embodiments, the nosecone 424 may be monolithically formed with the elongate shaft 420. The guidewire lumen 422 may extend proximally from the nosecone 424 within the elongate shaft 420. The nosecone 424 may be tapered radially inward in a distal direction.

In some embodiments, the elongate shaft 420 may comprise an inflation lumen 426 in fluid communication with the inflatable balloon 430. In some embodiments, the inflation lumen 426 may be disposed within the elongate shaft 420, as illustrated via broken line. In some alternative embodiments, the inflation lumen 426 may extend along an outer surface of the elongate shaft 420. Other configurations are also contemplated. In some embodiments, the inflation lumen 426 and/or the inflatable balloon 430 may be in fluid communication with a source of inflation fluid (not shown) disposed at and/or near a proximal end of the stent delivery system 410 and/or the elongate shaft 420. The proximal end of the stent delivery system 410 and/or the elongate shaft 420 may be disposed, held, and/or manipulated outside of a patient during a procedure to deploy the self-expanding endoprosthesis 300 within the body lumen 10.

The self-expanding endoprosthesis 300 may be configured to be disposed on the inflatable balloon 430 in the radially collapsed configuration with the inflatable balloon 430 in the deflated configuration. The self-expanding endoprosthesis 300 may be configured to be disposed around the inflatable balloon 430 in the radially collapsed configuration with the inflatable balloon 430 in the deflated configuration. The self-expanding endoprosthesis 300 may be configured to surround the inflatable balloon 430 in the radially collapsed configuration with the inflatable balloon 430 in the deflated configuration.

In some embodiments, the stent delivery system 410 may comprise an endoprosthesis retention element 440 disposed around (e.g., surrounding, etc.) the self-expanding endoprosthesis 300 in the radially collapsed configuration and the inflatable balloon 430 in the deflated configuration. In some embodiments, the endoprosthesis retention element 440 may comprise a tubular sleeve 442. Other configurations are also contemplated.

In some embodiments, the endoprosthesis retention element 440 and/or the tubular sleeve 442 may be disposed radially outward of and in intimate (e.g., direct) contact with the self-expanding endoprosthesis 300 in the radially collapsed configuration. In some embodiments, the endoprosthesis retention element 440 and/or the tubular sleeve 442 may be configured to constrain the self-expanding endoprosthesis 300 in the radially collapsed configuration on the inflatable balloon 430 when the inflatable balloon 430 is in the deflated configuration. In some embodiments, the self-expanding endoprosthesis 300 may be constrained on the inflatable balloon 430 in the radially collapsed configuration with the inflatable balloon 430 in the deflated configuration by the endoprosthesis retention element 440 and/or the tubular sleeve 442.

In some embodiments, the endoprosthesis retention element 440 and/or the tubular sleeve 442 may have an overall length 444 that is less than 125% of a length of the self-expanding endoprosthesis 300. In some embodiments, the endoprosthesis retention element 440 and/or the tubular sleeve 442 may have an overall length 444 that is less than 115% of a length of the self-expanding endoprosthesis 300. In some embodiments, the endoprosthesis retention element 440 and/or the tubular sleeve 442 may have an overall length 444 that is less than 110% of a length of the self-expanding endoprosthesis 300. In some embodiments, the endoprosthesis retention element 440 and/or the tubular sleeve 442 may have an overall length 444 that is less than 105% of a length of the self-expanding endoprosthesis 300. In some embodiments, the endoprosthesis retention element 440 and/or the tubular sleeve 442 may have an overall length 444 that is substantially equal to a length of the self-expanding endoprosthesis 300. In some alternative embodiments, the endoprosthesis retention element 440 and/or the tubular sleeve 442 may have an overall length 444 that is less than a length of the self-expanding endoprosthesis 300.

In some embodiments, the endoprosthesis retention element 440 and/or the tubular sleeve 442 is spaced apart from the proximal end of the stent delivery system 410 and/or the elongate shaft 420. In some embodiments, the endoprosthesis retention element 440 and/or the tubular sleeve 442 does not extend to the proximal end of the stent delivery system 410 and/or the elongate shaft 420. In some embodiments, the endoprosthesis retention element 440 and/or the tubular sleeve 442 is not fixedly attached directly to any other structure of the stent system 400 and/or the stent delivery system 410. In some embodiments, the endoprosthesis retention element 440 and/or the tubular sleeve 442 may be axially immovable relative to the self-expanding endoprosthesis 300 via manipulation of the stent delivery system 410. For example, movement of the elongate shaft 420 may fail to produce relative movement between the self-expanding endoprosthesis 300 and the endoprosthesis retention element 440 and/or the tubular sleeve 442.

In some embodiments, the stent delivery system 410 may be devoid of an actuation mechanism, such as but not limited to a pull wire, a filament, a thread, a shaft, a rod, a tube, a proximal extension, a finger grip, a mechanical assist, etc., configured to move the endoprosthesis retention element 440 and/or the tubular sleeve 442 axially relative to the self-expanding endoprosthesis 300. In some embodiments, the stent delivery system 410 may be devoid of an actuation mechanism, such as but not limited to a pull wire, a filament, a thread, a shaft, a rod, a tube, a proximal extension, a finger grip, a mechanical assist, etc., configured to move the endoprosthesis retention element 440 and/or the tubular sleeve 442 axially relative to the elongate shaft 420. In some embodiments, the stent delivery system 410 may be devoid of an actuation mechanism, such as but not limited to a pull wire, a filament, a thread, a shaft, a rod, a tube, a proximal extension, a finger grip, a mechanical assist, etc., configured to move the endoprosthesis retention element 440 and/or the tubular sleeve 442 axially relative to the elongate shaft 420 without moving the elongate shaft 420.

In some embodiments, the self-expanding endoprosthesis 300 may be configured to exert a first radially outward force against an inner surface of the endoprosthesis retention element 440 and/or the tubular sleeve 442 in the radially collapsed configuration. In some embodiments, the first radially outward force exerted by the self-expanding endoprosthesis 300 in the radially collapsed configuration may be insufficient and/or may be unable to overcome (e.g., break, fracture, rupture, shatter, etc.) the endoprosthesis retention element 440 and/or the tubular sleeve 442. In at least some embodiments, the first radially outward force exerted against the inner surface of the endoprosthesis retention element 440 and/or the tubular sleeve 442 by the self-expanding endoprosthesis 300 may be configured to secure the endoprosthesis retention element 440 and/or the tubular sleeve 442 in place over the self-expanding endoprosthesis 300 and the inflatable balloon 430. As such, the endoprosthesis retention element 440 and/or the tubular sleeve 442 may be configured to constrain and/or retain the self-expanding endoprosthesis 300 in the radially collapsed configuration absent additional radially outward force applied thereto.

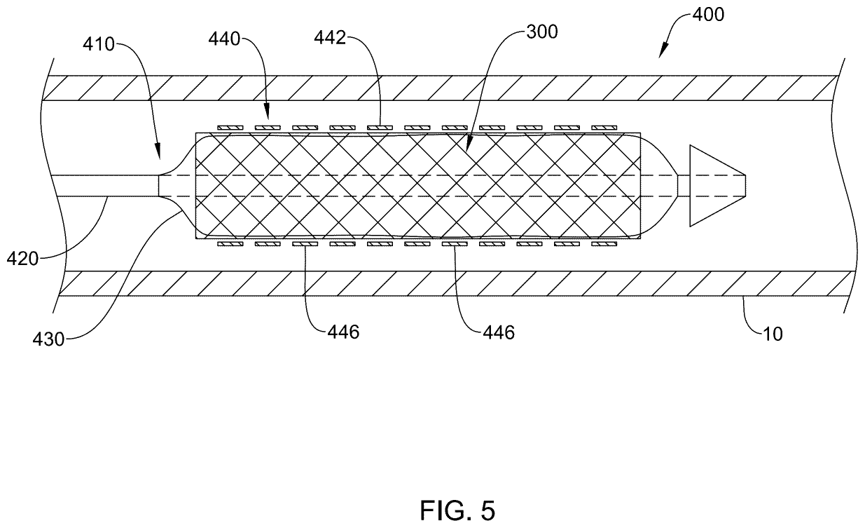

In some embodiments, an inner diameter of the endoprosthesis retention element 440 and/or the tubular sleeve 442 (e.g., FIG. 4) may be less than a radially outermost extent of the inflatable balloon 430 in the inflated configuration (e.g., FIGS. 5-7). In at least some embodiments, the inflatable balloon 430 may be non-compliant. For example, the inflatable balloon 430 may have a predefined shape in the inflated configuration. In some alternative embodiments, the inflatable balloon 430 may be compliant or partially compliant. In at least some embodiments, the inflatable balloon 430 may be formed from a polymeric material. Other configurations are also contemplated. Some suitable but non-limiting examples of materials for the inflatable balloon 430 are described below.

In some embodiments, the endoprosthesis retention element 440 and/or the tubular sleeve 442 may be configured to fracture, rupture, break apart, shatter, etc. when a second radially outward force greater than the first radially outward force is applied to the inner surface of the endoprosthesis retention element 440 and/or the tubular sleeve 442. In some embodiments, the inflatable balloon 430 may be configured to apply the second radially outward force to the inner surface of the endoprosthesis retention element 440 and/or the tubular sleeve 442 when the inflatable balloon 430 is in the inflated configuration. In some embodiments, the inflatable balloon 430 may be configured to apply the second radially outward force to the inner surface of the endoprosthesis retention element 440 and/or the tubular sleeve 442 when the inflatable balloon 430 is shifting and/or shifted from the deflated configuration toward and/or to the inflated configuration. In some embodiments, the endoprosthesis retention element 440 and/or the tubular sleeve 442 may be configured to fracture, rupture, break apart, shatter, etc. upon inflation of the inflatable balloon 430 toward and/or to the inflated configuration.

In some embodiments, the endoprosthesis retention element 440 and/or the tubular sleeve 442 may be configured to fracture, rupture, break apart, shatter, etc. into a plurality of fragments 446 when the inflatable balloon 430 is shifted from the deflated configuration toward and/or to the inflated configuration, as seen in FIG. 5. The plurality of fragments 446 may be discontinuous and/or unattached to each other. In some embodiments, the endoprosthesis retention element 440 and/or the tubular sleeve 442 may be configured to fracture, rupture, break apart, shatter, etc. along predetermined lines and/or predetermined fracture points into the plurality of fragments 446 when the inflatable balloon 430 is shifted from the deflated configuration toward and/or to the inflated configuration (e.g., the plurality of fragments 446 may be predetermined shapes, sizes, etc.). In some embodiments, the endoprosthesis retention element 440 and/or the tubular sleeve 442 may be configured to fracture, rupture, break apart, shatter, etc. randomly into the plurality of fragments 446 when the inflatable balloon 430 is shifted from the deflated configuration toward and/or to the inflated configuration (e.g., the plurality of fragments 446 may have random shapes, sizes, etc.). Other configurations, including combinations thereof, are also contemplated.

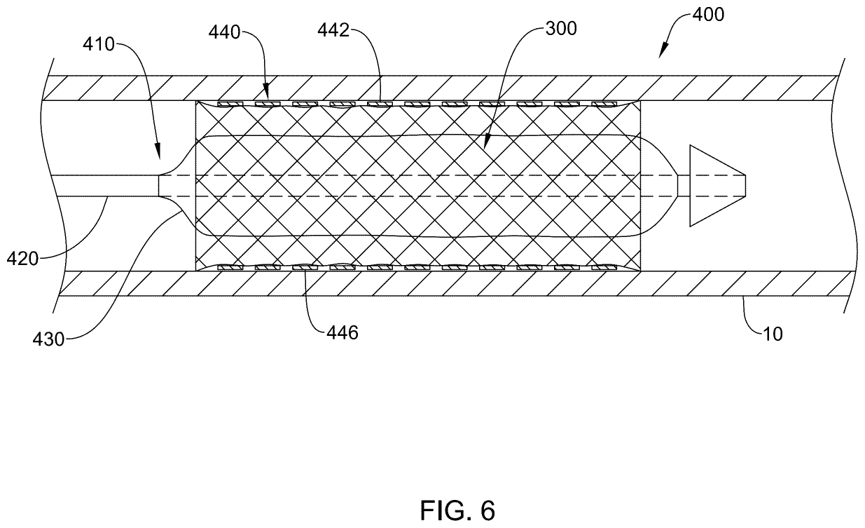

In some embodiments, the endoprosthesis retention element 440 and/or the tubular sleeve 442 may be configured to retain its structural integrity until the endoprosthesis retention element 440 and/or the tubular sleeve 442 is fractured, ruptured, broken apart, shattered, etc. as a result of shifting the inflatable balloon 430 from the deflated configuration (e.g., FIG. 4) toward and/or to the inflated configuration (e.g., FIG. 5). In some embodiments, fracturing the endoprosthesis retention element 440 and/or the tubular sleeve 442 may be configured to completely detach the endoprosthesis retention element 440 and/or the tubular sleeve 442 from a remainder of the stent delivery system 410, as seen in FIGS. 6 and 8.

Thereafter, the self-expanding endoprosthesis 300 may be configured and/or self-biased to shift toward and/or to the radially expanded configuration, as seen in FIG. 6, when the self-expanding endoprosthesis 300 is unconstrained by the endoprosthesis retention element 440 and/or the tubular sleeve 442. The self-expanding endoprosthesis 300 may push and/or urge the plurality of fragments 446 of the endoprosthesis retention element 440 and/or the tubular sleeve 442 radially outward against an inner wall of the body lumen 10. As such, the plurality of fragments 446 of the endoprosthesis retention element 440 and/or the tubular sleeve 442 may be configured to remain within the body lumen 10 after deploying the self-expanding endoprosthesis 300 and/or after shifting the self-expanding endoprosthesis 300 to the radially expanded configuration. Stated differently, the endoprosthesis retention element 440 and/or the tubular sleeve 442 is not removed from the body lumen 10 with the remainder of the stent delivery system 410 after the self-expanding endoprosthesis 300 has been deployed. Instead, after the endoprosthesis retention element 440 and/or the tubular sleeve 442 is fractured, ruptured, broken apart, shattered, etc., the inflatable balloon 430 may be deflated (e.g., shifted to the deflated configuration) and the self-expanding endoprosthesis 300 may shift toward and/or to the radially expanded configuration. In some embodiments, after the endoprosthesis retention element 440 and/or the tubular sleeve 442 is fractured, ruptured, broken apart, shattered, etc., the inflatable balloon 430 may be deflated (e.g., shifted to the deflated configuration) and the self-expanding endoprosthesis 300 may shift toward and/or to the radially expanded configuration simultaneously. after the endoprosthesis retention element 440 and/or the tubular sleeve 442 is fractured, ruptured, broken apart, shattered, etc., the inflatable balloon 430 may be deflated (e.g., shifted to the deflated configuration) and the self-expanding endoprosthesis 300 may shift toward and/or to the radially expanded configuration in sequence (e.g., one after the other).

In some embodiments, a radially outermost extent of the inflatable balloon 430 in the inflated configuration (e.g., FIGS. 5-6) may be less than a radially innermost extent of the self-expanding endoprosthesis 300 in the radially expanded configuration (e.g., FIGS. 8-9). In some embodiments, shifting the inflatable balloon 430 to the inflated configuration may not fully shift the self-expanding endoprosthesis 300 to the radially expanded configuration, as seen in FIG. 6. In some embodiments, the inflatable balloon 430 may be incapable of fully shifting the self-expanding endoprosthesis 300 to the radially expanded configuration. In some embodiments, the inflatable balloon 430 may be non-compliant and/or may have a pre-defined shape and/or size in the inflated configuration.

It should be noted that the use of a self-expanding endoprosthesis (ref. 300) with an inflatable balloon (ref. 430) is atypical as the self-expanding endoprosthesis 300 has no need for a secondary expansion means. In the stent system 400 of the disclosure, the inflatable balloon 430 is intended only to fracture, rupture, break apart, shatter, etc. the endoprosthesis retention element 440 and/or the tubular sleeve 442 into the plurality of fragments 446, which the self-expanding endoprosthesis 300 cannot do on its own. Thereafter, the self-expanding endoprosthesis 300 may be able and/or configured to shift to the radially expanded configuration on its own.

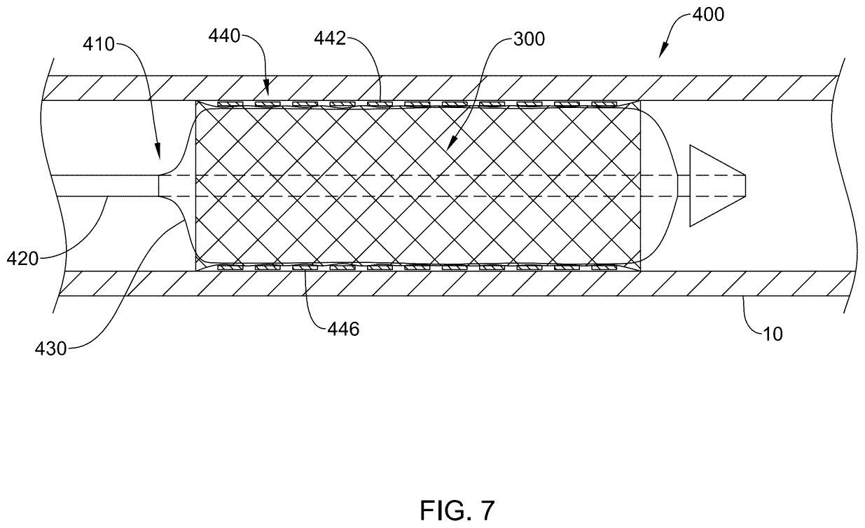

In some alternative embodiments, shifting the inflatable balloon 430 to the inflated configuration may aid in fully shifting the self-expanding endoprosthesis 300 to the radially expanded configuration, as seen in FIG. 7. For example, the inflatable balloon 430 in FIG. 7 may have a radially outermost extent in the inflated configuration that is greater than the radially outermost extent of the inflatable balloon 430 shown in FIG. 6.

After shifting the self-expanding endoprosthesis 300 to the radially expanded configuration, the remainder of the stent delivery system 410 may be removed from the body lumen 10, as seen in FIG. 8, thereby leaving the plurality of fragments 446 of the endoprosthesis retention element 440 and/or the tubular sleeve 442 trapped between the inner wall of the body lumen 10 and the self-expanding endoprosthesis 300. The plurality of fragments 446 of the endoprosthesis retention element 440 and/or the tubular sleeve 442 may be trapped against the inner wall of the body lumen 10 by the self-expanding endoprosthesis 300.

In at least some embodiments, the endoprosthesis retention element 440 and/or the tubular sleeve 442 (and/or the plurality of fragments 446 thereof) may be formed from a biocompatible material. In some embodiments, the endoprosthesis retention element 440 and/or the tubular sleeve 442 (and/or the plurality of fragments 446 thereof) may be bioabsorbable and/or may be formed from a bioabsorbable material. As such, the endoprosthesis retention element 440 and/or the tubular sleeve 442 (and/or the plurality of fragments 446 thereof) may be configured to be absorbed by the body lumen 10 and/or fluid (e.g., blood, etc.) flowing through the body lumen 10, as seen in FIG. 9. Some suitable but non-limiting examples of materials for the endoprosthesis retention element 440 and/or the tubular sleeve 442 (and/or the plurality of fragments 446 thereof) are described below.

The materials that can be used for the various components of the endoprosthesis and/or the endoprosthesis system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the delivery device, the endoprosthesis, the single wire, the polymeric covering, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni-Co-Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A stent system, comprising:
a self-expanding endoprosthesis configured to shift from a radially collapsed configuration to a radially expanded configuration; and
a stent delivery system, comprising:
an elongate shaft comprising an inflatable balloon disposed proximate a distal end of the elongate shaft, wherein the inflatable balloon is configured to shift between a deflated configuration and an inflated configuration;
wherein the self-expanding endoprosthesis is configured to be disposed on the inflatable balloon in the radially collapsed configuration with the inflatable balloon in the deflated configuration; and
an endoprosthesis retention element disposed around the self-expanding endoprosthesis in the radially collapsed configuration and the inflatable balloon in the deflated configuration;
wherein the endoprosthesis retention element is configured to constrain the self-expanding endoprosthesis in the radially collapsed configuration;
wherein the endoprosthesis retention element is configured to fracture upon inflation of the inflatable balloon to the inflated configuration.

2. The stent system of claim 1, wherein the endoprosthesis retention element has an overall length that is less than 125% of a length of the self-expanding endoprosthesis.

3. The stent system of claim 1, wherein a radially outermost extent of the inflatable balloon in the inflated configuration is less than a radially innermost extent of the self-expanding endoprosthesis in the radially expanded configuration.

4. The stent system of claim 1, wherein shifting the inflatable balloon to the inflated configuration does not shift the self-expanding endoprosthesis to the radially expanded configuration.

5. The stent system of claim 1, wherein an inner diameter of the endoprosthesis retention element is less than a radially outermost extent of the inflatable balloon in the inflated configuration.

6. The stent system of claim 1, wherein the endoprosthesis retention element does not extend to a proximal end of the elongate shaft.

7. The stent system of claim 1, wherein the stent delivery system is devoid of an actuation mechanism configured to move the endoprosthesis retention element axially relative to the elongate shaft without moving the elongate shaft.

8. A stent system, comprising:
a self-expanding endoprosthesis configured to shift from a radially collapsed configuration to a radially expanded configuration, wherein the self-expanding endoprosthesis is configured to exert a first radially outward force in the radially collapsed configuration; and
a stent delivery system, comprising:
an elongate shaft comprising an inflatable balloon disposed proximate a distal end of the elongate shaft, wherein the inflatable balloon is configured to shift between a deflated configuration and an inflated configuration;
wherein the self-expanding endoprosthesis is configured to be disposed on the inflatable balloon in the radially collapsed configuration with the inflatable balloon in the deflated configuration; and
an endoprosthesis retention element disposed around the self-expanding endoprosthesis in the radially collapsed configuration and the inflatable balloon in the deflated configuration;
wherein the endoprosthesis retention element is configured to constrain the self-expanding endoprosthesis in the radially collapsed configuration;
wherein the endoprosthesis retention element is configured to fracture when a second radially outward force greater than the first radially outward force is applied to an inner surface of the endoprosthesis retention element.

9. The stent system of claim 8, wherein the endoprosthesis retention element is bioabsorbable.

10. The stent system of claim 8, wherein fracturing the endoprosthesis retention element is configured to completely detach the endoprosthesis retention element from a remainder of the stent delivery system.

11. The stent system of claim 8, wherein the stent delivery system is devoid of an actuation mechanism configured to move the endoprosthesis retention element axially relative to the self-expanding endoprosthesis.

12. The stent system of claim 8, wherein the inflatable balloon is configured to apply the second radially outward force against the inner surface of the endoprosthesis retention element.

13. The stent system of claim 12, wherein the inflatable balloon is configured to apply the second radially outward force against the inner surface of the endoprosthesis retention element when the inflatable balloon is in the inflated configuration.

14. The stent system of claim 8, wherein the inflatable balloon is incapable of fully shifting the self-expanding endoprosthesis to the radially expanded configuration.

15. A stent system, comprising:
a self-expanding endoprosthesis configured to shift from a radially collapsed configuration to a radially expanded configuration; and
a stent delivery system, comprising:
an elongate shaft comprising an inflatable balloon disposed proximate a distal end of the elongate shaft, wherein the inflatable balloon is configured to shift between a deflated configuration and an inflated configuration;
wherein the self-expanding endoprosthesis is configured to be disposed on the inflatable balloon in the radially collapsed configuration with the inflatable balloon in the deflated configuration; and
an endoprosthesis retention element comprising a tubular sleeve disposed radially outward of and in intimate contact with the self-expanding endoprosthesis, wherein the endoprosthesis retention element is configured to constrain the self-expanding endoprosthesis in the radially collapsed configuration on the inflatable balloon when the inflatable balloon is in the deflated configuration;
wherein the tubular sleeve is configured to break apart when the inflatable balloon is shifted from the deflated configuration toward the inflated configuration.

16. The stent system of claim 15, wherein the tubular sleeve is axially immovable relative to the self-expanding endoprosthesis via manipulation of the stent delivery system.

17. The stent system of claim 15, wherein the tubular sleeve is not fixedly attached directly to any other structure of the stent system.

18. The stent system of claim 15, wherein the self-expanding endoprosthesis is formed from a single filament of material.

19. The stent system of claim 15, wherein the tubular sleeve is configured to break apart into a plurality of fragments when the inflatable balloon is shifted from the deflated configuration toward the inflated configuration.

20. The stent system of claim 15, wherein the tubular sleeve is configured to retain its structural integrity until the tubular sleeve is broken apart as a result of shifting the inflatable balloon from the deflated configuration toward the inflated configuration.
